(19) United States
(12) Patent Application Publication
(10) Pub. No.: US 2004/0072857 A1
(43) Pub. Date: Apr. 15, 2004

(54) Polymerized and modified rapamycins and their use in coating medical prostheses

(76) Inventors: Jacob Waugh, Palo Alto, CA (US); Mahmood K. Razavi, San Carlos, CA (US); Camran Nezhat, Woodside, CA (US); Pamela N. Cifra, Daly City, CA (US); Michael D. Dake, Stanford, CA (US)

Correspondence Address: MANATT PHELPS AND PHILLIPS, ROBERT D. BECKER, 1001 PAGE MILL ROAD, BUILDING 2, PALO ALTO, CA, 94304, US

(21) Appl. No.: 10/613,584

(22) Filed: Jul. 2, 2003

Related U.S. Application Data
(60) Provisional application No. 60/393,686, filed on Jul. 2, 2002.

(51) Int. Cl. A61F 2/06; A61K 31/4745

(52) U.S. Cl. 514/291; 623/1.46

(57) ABSTRACT

Compositions of matter comprise linked pluralities of mTOR-binding molecules, such as rapamycin. The compositions may be directly polymerized or may comprise rapamycin or other mTOR-binding molecules covalently or non-covalently attached to a backbone molecule. The compositions may be bound to vascular prostheses and other implantable devices in order to inhibit hyperplasia or for other therapeutic purposes.

### REFERENCE TO PRIOR APPLICATION

This application for patent claims, under 35 U.S.C. § 119(e), the benefits of the filing date of U.S. Provisional Application Serial No. 60/393,686, entitled “Polymerized and Modified Rapamycins and their use in Coating Medical Prostheses” and filed on Jul. 2, 2002

### BACKGROUND OF THE INVENTION

1. Field of the Invention

The present invention relates generally to medical devices and methods. More particularly, the present invention relates to novel forms of rapamycin and related compositions which are useful for coating medical prostheses.

Coronary artery disease is the leading cause of death and morbidity in the United States and other western societies. In particular, atherosclerosis in the coronary arteries can cause myocardial infarction, commonly referred to as a heart attack, which can be immediately fatal or, even if survived, can cause damage to the heart which can incapacitate the patient.

While coronary artery bypass surgery is an effective treatment for stenosed arteries resulting from arthrosclerosis and other causes, it is a highly invasive procedure which is also expensive and which requires substantial hospital and recovery time. Percutaneous transluminal angioplasty (PTCA), commonly referred to as balloon angioplasty, is less invasive, less traumatic, and significantly less expensive than bypass surgery. Until recently, however, balloon angioplasty has not been considered to be as effective a treatment as bypass surgery. The effectiveness of balloon angioplasty, however, has improved significantly with the introduction of stenting which involves the placement of a scaffold structure within the artery which has been treated by balloon angioplasty. The stent inhibits abrupt reclosure of the artery and has some benefit in inhibiting subsequent restenosis resulting from hyperplasia.

Despite this benefit, patients who have undergone angioplasty procedures with subsequent stenting still suffer from a high incidence of restenosis resulting from hyperplasia. Very recently, however, experimental trials have demonstrated that the coating of stents with anti-proliferative drugs, such as rapamycin, can significantly reduce the occurrence of hyperplasia, promising to make combined angioplasty and stenting a viable alternative to bypass surgery.

While holding great promise, the ability of rapamycin and related compounds to inhibit hyperplasia is limited by the ability to bind effective amounts and concentrations of the drug onto the surface of the stent or other vascular prosthesis being used. In particular, the ability to load rapamycin and related compounds is limited by the nature of the surface of the stent or other vascular prosthesis to which the rapamycin is to be bound, typically a stainless steel surface. Such stainless steel and other prosthetic surfaces are usually limited in the number of reactive sites available for covalent or other attachment of the rapamycin. Binding under such circumstances usually results in a low dose of the rapamycin and/or a restricted time course of release of the rapamycin. Both these circumstances can dramatically limit the efficiency of the rapamycin.

In an effort to enhance the effectiveness of rapamycin and other anti-proliferative drugs bound to stents, a common approach has been to entrap the drugs in a polymer matrix which is coated or otherwise disposed over the stent surface. While the use of such a polymer matrix may increase the amount of drug and/or provide for desired controlled release characteristics, such passive containment of the drug has a number of critical limitations. First, the polymers used in the matrix will inevitably exert some biologic effect. For example, biodegradable polymers, such as PLGA (a co-polymer of glycolic acid and lactic acid), when hydrolyzed in a vascular environment may cause a strong local reduction in pH (due to the release of the acidic monomers) which can have a deleterious effect on the treatment. Moreover, if the polymers of the matrix are not themselves anchored to the stent surface, the polymers can fragment or embolize, adversely affecting the release characteristics of matrix and having a potentially direct harmful effect on the patient.

For these reasons, improved compositions, methods, and apparatus for the coating and release of rapamycin and related drugs from the surface of vascular and other prostheses would be desirable. In particular, it would be desirable to avoid the use of polymer matrices for passively sequestering the drug. It would be further desirable to provide for the direct covalent or non-covalent binding of large amounts of the rapamycin or related drug directly to the surface of the prostheses. It would still further be desirable if the rapamycin or related drug could itself be polymerized or linked to a backbone polymer in order to enhance the amounts of drug available and simplify binding of the enhanced drug amounts to the surface of the prostheses. At least some of these objectives will be met by the inventions described hereinafter.

2. Description of the Background Art

Vascular stents coated with rapamycin and related drugs are described in U.S. Pat. Nos. 6,369,039; 6,368,658; 6,273,913, 6,153,252; and 5,516,781. Rapamycin analogues and related drugs are described in U.S. Pat. Nos. 6,329,386 and 6,313,264. Derivative forms of rapamycin are described in a number of patents including U.S. Pat. Nos. 5,985,890; 5,780;462; 5,504,091; 5,411,967; 5,391,730; 5,389,639; 5,385,909; 5,385,908; 5,362,718; 5,302,584; 5,258,389; 5,233,036; 5,221,670; 5,177,203; 5,162,333; 5,151,413; 5,130,307; 5,120,842, 5,118,678; 5,118,677; 5,100,883; 5,023,264; 4,650,803; 4,401,653; and 4,316,885. Binding of rapamycin to polyethylene glycol to enhance solubility is described in U.S. Pat. Nos. 5,516,770; 5,530,006; and 6,331,547; U.S. Patent Publication No. 2002/005518 A1; and PCT Publications WO 02/24706 and WO 01/126633. The full disclosures of each reference cited herein is incorporated by reference for all purposes.

### BRIEF SUMMARY OF THE INVENTION

The present invention provides improved compositions of matter and methods for their preparation and use for coating medical devices, such as stents, grafts, and other vascular prostheses. The improved compositions of matter comprise linked pluralities of molecules which specifically bind to the mammalian target of rapamycin (mTOR). Such molecules include rapamycin as well as analogues and derivatives of rapamycin, such as CCI-779, RAD-001, SDZ Rad (Everolimus), (FK506) Tacrolimus, ASM 981 (Pimecrolimus), Wortmannin and Tumistatin.

Rapamycin, also called sirolimus, is a peptide having the chemical formula C₅₁H₇₉NO₁₃ and a molecular weight of 913. The structure of rapamycin is shown in FIG. 1, where the structure has been annotated to show the binding regions and hydroxyl at position 42, as discussed hereinbelow. The rapamycin peptide was first isolated from Streptomyces hygroscopicus. Rapamycin is an immunosuppressant which is currently approved for the treatment of organ transplant patients and is available under the tradename RAPAMUNE™ from American Home Products. As discussed above, rapamycin also shows great promise as an anti-hyperplastic agent for coating for stents, grafts, and other vascular prostheses.

As used herein, the phrase “molecule which specifically binds to the mammalian target of rapamycin (mTOR)” shall include all known and yet to be discovered compounds which both bind to the mTOR and which have immunosuppression and/or anti-hyperplastic activity which is similar to that of rapamycin. The term “rapamycin” refers more specifically to rapamycin as well as known and yet to be discovered analogues and derivatives of rapamycin which have an analogous, but modified, structure to that of native rapamycin. The phrase “mTOR-binding compounds” and “mTOR-binding drugs” will refer collectively to rapamycin and all such related compounds, including analogues and derivatives of rapamycin.

Compositions of matter according to the present invention comprise a linked plurality of mTOR-binding molecules, including rapamycin and analogues and derivatives thereof, as defined above. The molecules may be linked directly or indirectly. When the mTOR-binding molecules are linked directly, they will be referred to as being polymerized. Indirect linking of the molecules will typically occur through backbone molecule(s), where the rapamycin or other molecules may be covalently or non-covalently attached to the backbone molecule(s). Exemplary backbones include poly (amino acids), polyalcohols, nucleic acids, sphingosine, polysaccharides, polyamines, hydroxyaliphatic carboxylic acids, and other homo- or copolymers with active side chains, such as carboxylates, amines, hydroxyls, and the like, which may serve as binding moieties as described below. Such backbone molecule(s) may be linear or branched, and more than one backbone molecule may be present in the linked plurality of molecules. That is, two or more backbone molecules may be joined non-covalently or otherwise be associated to form a single linked plurality of mTOR-binding molecules.

The number of rapamycin or other molecules present in the linked plurality of molecules of the present invention may vary widely, typically being from as few as 3 to as many as 10⁶ molecules, more usually being from 5 to 10⁵ molecules, and still more usually being from 7 to 5×10⁴ molecules, and often being in the range from 7 to 10³.

In a first exemplary embodiment, compositions according to the present invention comprise rapamycin molecules which have been derivatized with linking moieties, wherein the rapamycin molecules are covalently bound through the moieties to a backbone. Specific moieties are described in the Examples hereinbelow. The linking moieties may be heterobifunctional linkers which are reacted with the mTOR-binding molecules and the backbone molecule(s) or may result from a reaction between native or introduced groups or side chains on the mTOR-binding molecules and the backbone molecule(s). The linking moieties may be bound to the rapamycin molecules at sites which do not sterically interfere with active sites of rapamycin. In such cases, the rapamycin will retain its activity when attached to the backbone. In other instances, the linking moieties will be bound to the rapamycin molecules at sites which sterically or otherwise interfere with the active site(s) of rapamycin. In such cases, the rapamycin activity will be inhibited so long as the rapamycin remains attached to the backbone. Rapamycin activity will be restored as the rapamycin is released from the backbone, either by degradation of the backbone or lysing of the linking moieties. In the former case, compositions which remain stable and do not degrade when present in the vascular environment could find use. In the latter case, it would be necessary that the rapamycin be released from the backbone, e.g., where the backbone degrades under preselected conditions, and/or the linking moieties lyse under preselected conditions, typically characteristic of the vascular environment.

In other instances, ascorbic acid and other moieties may be bound to the rapamycin molecules and remain unlinked in the final linked plurality of molecules. The unlinked ascorbic acid or other moiety will preferably retain its native activity, e.g., as an antioxidant, in the final composition.

Rapamycin's properties, in part, are dependent on its binding to FKBP12 (FK506 binding protein). The FKBP binding domain of rapamycin contains a pipecolinyl ring that binds in the hydrophobic pocket of FKBP (Sehgal S. (1998) Rapamune (RAPA, rapamycin, sirolimus): Mechanism of action immunosuppressive effect results from blockade of signal transduction and inhibition of cell cycle progression. Clin Biochem 31: 335-340). A 133 amino acid hydrophobic FKBP:RAPA binding domain (FRB) located upstream of the mammalian target of rapamycin sequence has a critical Ser 2035 residue whose mutation abolishes the RAPA:FKBP binding activity (Chen J et al. (1995) Identification of an 11 kDa FKBP12-rapamycin-binding domain within the 289 kDa FKBP12-rapamycin-associated protein and characterization of a critical serine residue. PNAS 92: 4947-51; Lorenz M C et al. (1995) TOR mutations confer rapamycin resistance by preventing interaction with FKBP12-rapamycin. J Biol Chem 270: 27531-7). Interaction of rapamycin with mTOR occurs through close contact with aromatic residues in the FRB region (Sehgal 1998).

The backbone molecule may comprise either a single molecule or a group of two or more non-covalently attached or otherwise associated molecules. The backbone molecule(s) will have sufficient size to carry the plurality of rapamycin or other mTOR-binding molecules as well as having the ability to covalently or non-covalently attach the rapamycin or other molecules. Thus, the backbone molecules will typically be polymers having molecular weights in the range from 93 to 10⁶, usually from 135 to 5×10⁵, more usually from 156 to 5×10⁴, and most usually from 177 to 2.5×10⁴. Suitable polymers include poly (amino acids), polyalcohols, nucleic acids, sphingosine, polysaccharides, polyamines, hydroxyaliphatic carboxylic acids, and other homo- or copolymers with active side chains, such as carboxylates, amines, hydroxyls, and the like, which may serve as binding moieties as described below. Poly (amino acids) will generally be preferred over proteins since the binding characteristics will be very uniform and depend on the nature of the specific amino acid incorporated.

The present invention further comprises implantable prostheses which are coated, covered, or otherwise associated with the compositions described above. In particular, the implantable prostheses will usually comprise a structure having a surface with linked pluralities of molecules which specifically bind to the mTOR, as described above, present on the surface. The structure may be any device which is implantable into humans or animals, including pacemakers, shunts, bypass grafts, drug delivery pumps, orthopedic implants, and the like. The present invention will find its greatest use with implantable vascular prostheses, such as vascular stents and grafts. Such vascular stents and grafts usually comprise an expandable metal scaffold which is implanted into a blood vessel in order to maintain patency of the blood vessel (when treating occlusive diseases) or to strengthen the blood vessel wall (when treating aneurysms).

In at least most instances, it will be desirable to coat or treat such vascular implantable prostheses with the compositions of the present invention in order to inhibit hyperplasia. Hyperplasia inhibition is of particular desirability when treating patients for atherosclerotic disease in the arteries, particularly the coronary arteries. In such instances, the patient will usually be treated initially with a recanalization procedure, usually balloon angioplasty (PTCA), but in other instances atherectomy, laser angioplasty, or the like, might find use. Following the initial recanalization procedure, a stent will usually be implanted in order to prevent abrupt reclosure and limit subsequent hyperplasia. By coating, depositing, or otherwise associating the compositions of the present invention on the stent, hyperplasia can be further inhibited.

The present invention can be used with virtually any stent or vascular graft structure. Most commonly, compositions of the present invention will be covalently attached to the available metal surfaces of the stent, as described in more detail below. The compositions may, however, be immobilized or sequestered on the stent in a variety of other ways. For example, they may be incorporated into channels or reservoirs formed on the surface of the stent. Optionally, they may be maintained in such reservoirs which are then covered with a controlled release membrane. Usually, however, they will not be incorporated directly into polymer matrices which release the compositions as they degrade. That is, it is a particular advantage of the present invention that large effective amounts of the rapamycin or other mTOR-binding substance may be bound directly to a surface of the prosthesis without the need to incorporate it within a carrier matrix.

The present invention still further comprises methods for preparing linked pluralities of molecules which specifically bind to mTOR. Generally, the methods comprise providing a backbone molecule and binding the plurality of mTOR-binding molecules to the backbone molecule. The mTOR-binding molecules will generally be as described above, and the molecules will be bound to the backbone in the amounts described above.

The present invention still further comprises methods for preparing linked pluralities of molecules which specifically bind to mTOR, when the methods comprise polymerizing the molecules. Usually, the mTOR-binding molecules will be derivatized with a linking moiety, and polymerization will be effected between the derivatized linking moiety and another moiety native to rapamycin or other mTOR-binding molecule. Alternatively, the polymerization may be directly between derivatized linking moieties present on each molecule. The mTOR-binding molecules and the numbers of molecules in each plurality of molecules will generally be as described above.

The present invention still further provides methods for modifying an implantable prosthesis with the compositions of the present invention. The implantable prosthesis may be any of the types described above. The methods comprise providing the prosthesis and binding linked mTOR-binding molecules to a surface of the prosthesis. In a preferred aspect of this method, a surface of the prosthesis, generally a metal surface, will be treated to generate free amines. Rapamycin or other mTOR-binding molecule will then be linked to the amine via a carboxy moiety which has been introduced to the rapamycin or is naturally present in other mTOR-binding molecule. The nature of the bound compositions is generally as described above.

### BRIEF DESCRIPTION OF THE DRAWINGS

FIG. 1 illustrates the structure of rapamycin with 42 hydroxyl indicated and binding portions bounded.

FIG. 2 is a schematic illustration of a first embodiment of the composition of the present invention where the rapamycin or other mTOR-binding molecule is attached to a backbone molecule.

FIG. 3 is a schematic illustration of a second embodiment of the compositions of the present invention where rapamycin or other mTOR-binding molecules are directly polymerized in a chain.

FIG. 4 is a block diagram illustrating a general preparation protocol for the compositions of FIG. 2.

FIG. 5 is a block diagram illustrating a specific preparation protocol for the compositions of FIG. 2 where the backbone molecule is a poly (amino acid).

FIG. 6 is a preparation protocol for a polymerized rapamycin composition as illustrated in FIG. 3. FIG. 7 illustrates a stent surface to be modified by the methods of the present invention.

FIGS. 8-11 illustrate exemplary protocols for binding the compositions of the present invention to the stent surface of FIG. 7.

FIGS. 12-16 illustrate exemplary protocols for derivatizing and/or linking rapamycin to produce the compositions of the present invention.

### DETAILED DESCRIPTION OF THE INVENTION

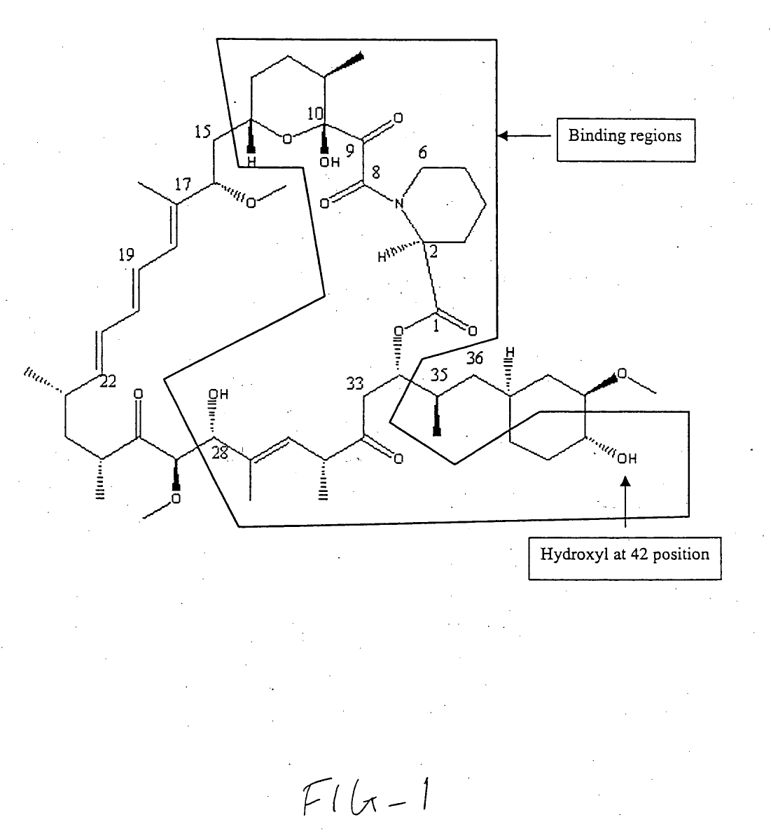

The following compositions and preparation protocols will be described with reference to rapamycin as the exemplary mTOR-binding molecule. It will be appreciated, however, that other mTOR-binding molecules may be incorporated into the compositions of the present invention using generally the same preparation protocols as described hereinafter. The structure of rapamycin is shown in FIG. 1.

Rapamycin's properties, in part, are dependent on its binding to FKBP12 (FK506 binding protein). The FKBP binding domain of rapamycin contains a pipecolinyl ring that binds in the hydrophobic pocket of FKBP (Sehgal S. (1998) Rapamune (RAPA, rapamycin, sirolimus): Mechanism of action immunosuppressive effect results from blockade of signal transduction and inhibition of cell cycle progression. Clin Biochem 31: 335-340). A 133 amino acid hydrophobic FKBP:RAPA binding domain (FRB) located upstream of the mammalian target of rapamycin sequence has a critical Ser 2035 residue whose mutation abolishes the RAPA:FKBP binding activity (Chen J et al. (1995) Identification of an 11 kDa FKBP12-rapamycin-binding domain within the 289 kDa FKBP12-rapamycin-associated protein and characterization of a critical serine residue. PNAS 92: 4947-51; Lorenz M C et al. (1995) TOR mutations confer rapamycin resistance by preventing interaction with FKBP 12-rapamycin. J Biol Chem 270: 27531-7). Interaction of rapamycin with mTOR occurs through close contact with aromatic residues in the FRB region (Sehgal 1998).

Referring now to FIG. 2, a first embodiment of the compositions of the present invention comprises a linked plurality of rapamycin molecules 10 where the individual rapamycin molecules 12 are covalently or non-covalently attached to at least one backbone molecule 14. Usually, the rapamycin molecules 12 will be derivatized with a linking moiety 16 which may be any of the molecules described above and which are preferably attached to the rapamycin molecules at the sites identified in the description of FIG. 1 above.

Useful and exemplary backbone molecules 14 have been described above. Particular protocols for covalently attaching rapamycin molecules to poly (amino acids) and polyalcohols are described below.

In general, the rapamycin molecules 12 will be linked to the backbone molecule 14 by linking molecules 16 which may be heterobifunctional molecules, i.e., molecules having different functional groups which are capable of selectively reacting at target sites on the rapamycin and separately at target sites on the backbone molecule 14. Thus, the particular linking molecule chosen will depend both on the linking site chosen on rapamycin as well as on the nature of the binding sites available on the backbone molecule. The linking molecules may be separate molecules which are reacted with both the rapamycin and the backbone molecule, typically being heterobifunctional agents. Alternatively, reactive groups or moieties on the rapamycin and the backbone molecule may be directly reacted to form a linkage. Typical linking molecules incorporated by both of these approaches are set forth in Table 1 below.

TABLE 1

| LINKING | RAPAMYCIN 12 | BACKBONE 14 |
|---|---|---|
| MOLECULE 16 | BINDING SITE | BINDING GROUP |
| Direct esterification | Hydroxyl 42 | Carbocxylic acid |
| PMPI¹ | Hydroxyl 42 | Thiol |
| Carbonic acid, | Hydroxyl 42 | Amine |
| bicarbonate, CO₂, | | |
| diacid, triacid (e.g., | | |
| citric acid), or other | | |
| multiacids | | |
| Disulfide | Thiol² | Thiol |
| Ester | Hydroxyl 42 | Hydroxyl |

¹Pierce-Endogen
²Thiols introduced by, for example, esterification with cystine of other free sulfhydryl-containing compounds.

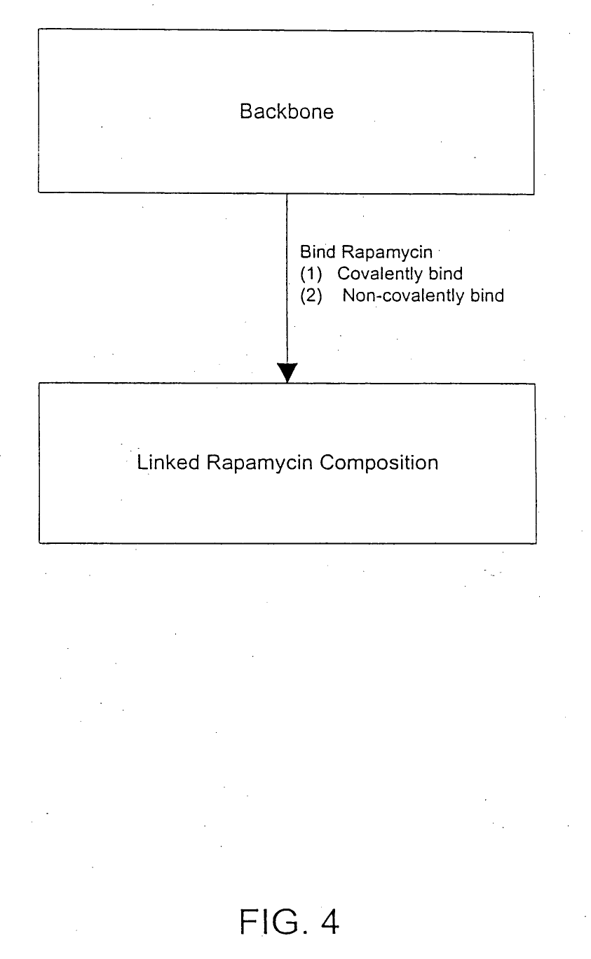

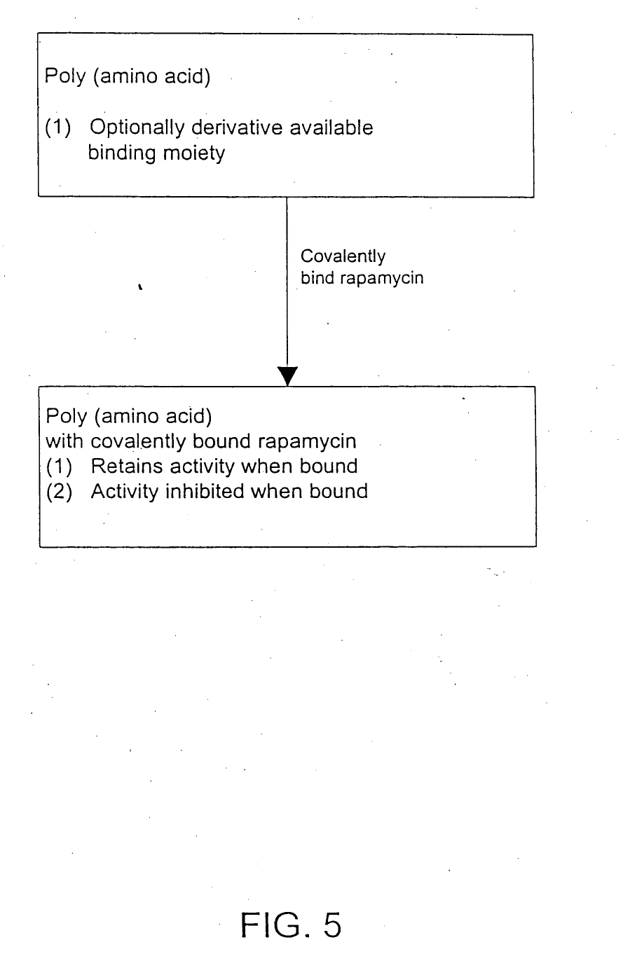

Generally, the compositions shown in FIG. 2 will be prepared by protocols as set forth in FIG. 4. The rapamycin will be covalently or non-covalently bound to the backbone to produce the linked rapamycin compositions of the present invention. A preferred protocol for preparing the compositions of the present invention where the backbone molecule is a poly (amino acid) is illustrated in FIG. 5. The rapamycin molecules are bound to the poly (amino acid) either through groups or moieties which are naturally present or which have been introduced to the poly (amino acid). Similarly, the rapamycin may be reacted with the poly (amino acid) either in its native form or after it has been derivatized to introduce one or more binding groups. A number of the exemplary preparation protocols rely on reacting the hydroxyl at position 42 of rapamycin (FIG. 1). Additionally, thiols, sulfhydryls, hydroxyls, carbonyls, carboxylates, amines, olefins, epoxides, halides, or other reactive functionalities with variable spacer arms, can be introduced to the hydroxyl at position 42 or elsewhere in the rapamycin molecule. The rapamycin may be bound to the backbone molecule either at positions which interfere with the rapamycin activity, or which not do not interfere with the rapamycin activity, as generally indicated in FIG. 1 above. When bound so that they sterically interfere with the active site, it will usually be necessary to release the rapamycin molecules from the linked plurality in order to achieve the desired inhibition of hyperplasia or other activity. When bound remotely from the active sites so that the active sites remain available, it will usually not be necessary to release the rapamycin in order to achieve the desired therapeutic or other benefit. In the latter cases, the compositions may be stable under the conditions of use. That is, there will be no need that the rapamycin molecules be released from either the compositions or the prostheses which they coat.

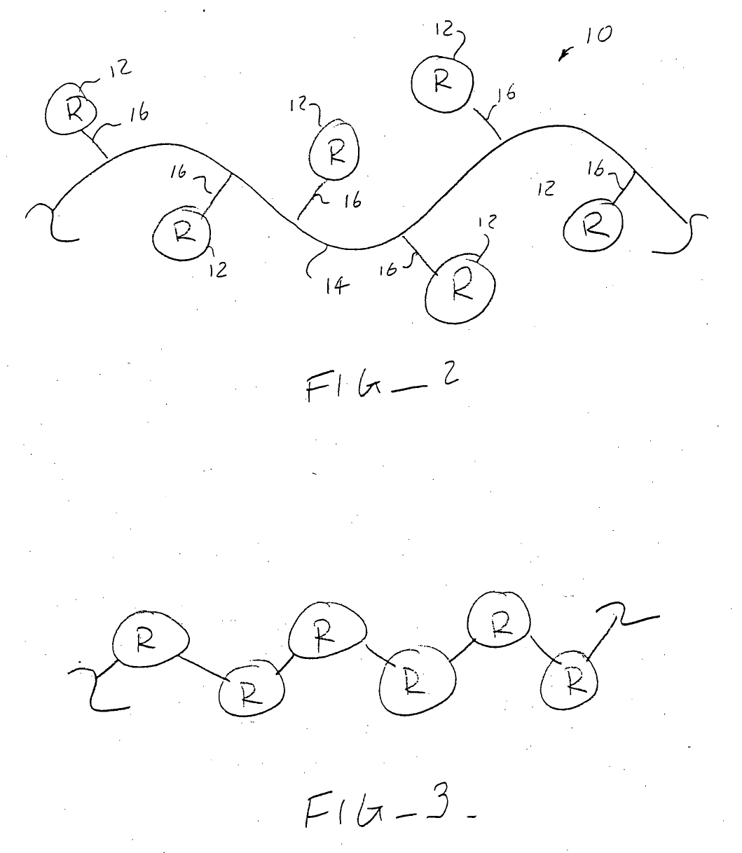

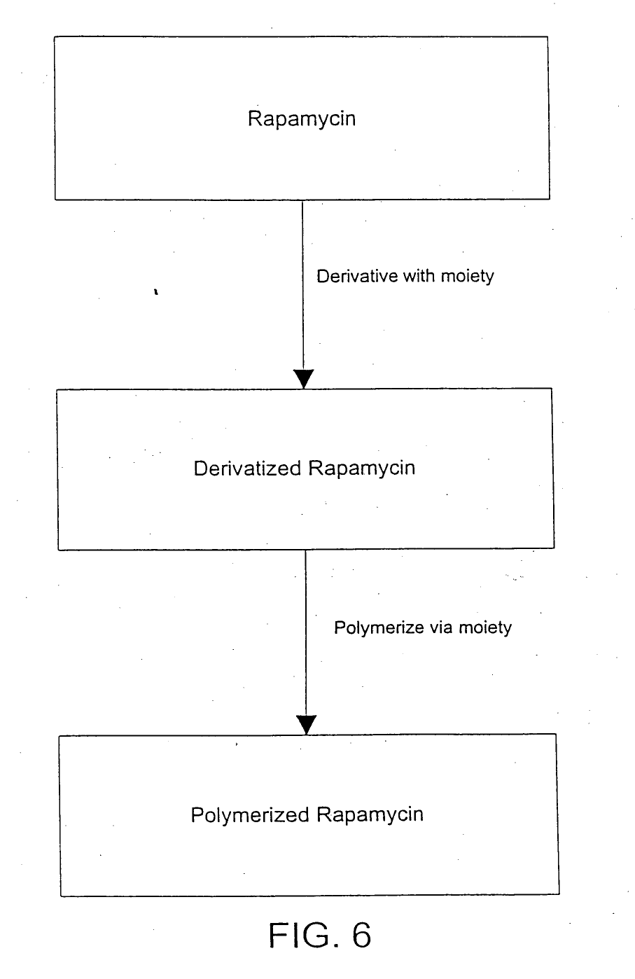

Preparation of the compositions of FIG. 3 are schematically illustrated in FIG. 6. Usually, the rapamycin will be derivatized with a moiety which permits polymerization. The derivatized rapamycin will then be polymerized via the moiety. Exemplary moieties which can be utilized for such polymerization techniques include ascorbic acid, citric acid, aspartate, glutamate, and the like. Exemplary binding sites for these moieties are found at both hydroxyls of rapamycin, particularly at the hydroxyl on position 42 as indicated in FIG. 1.

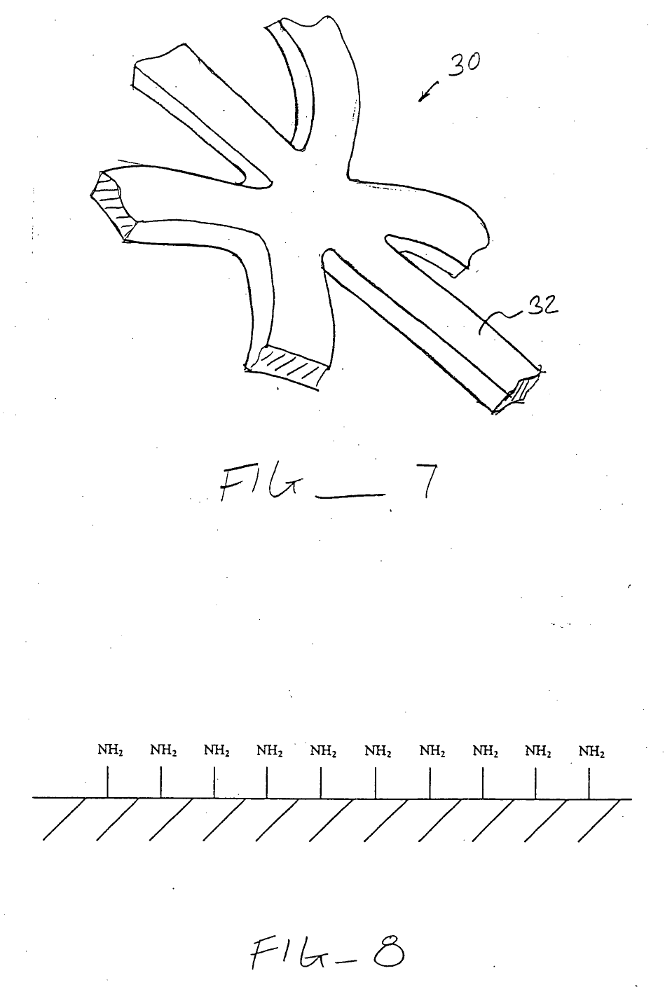

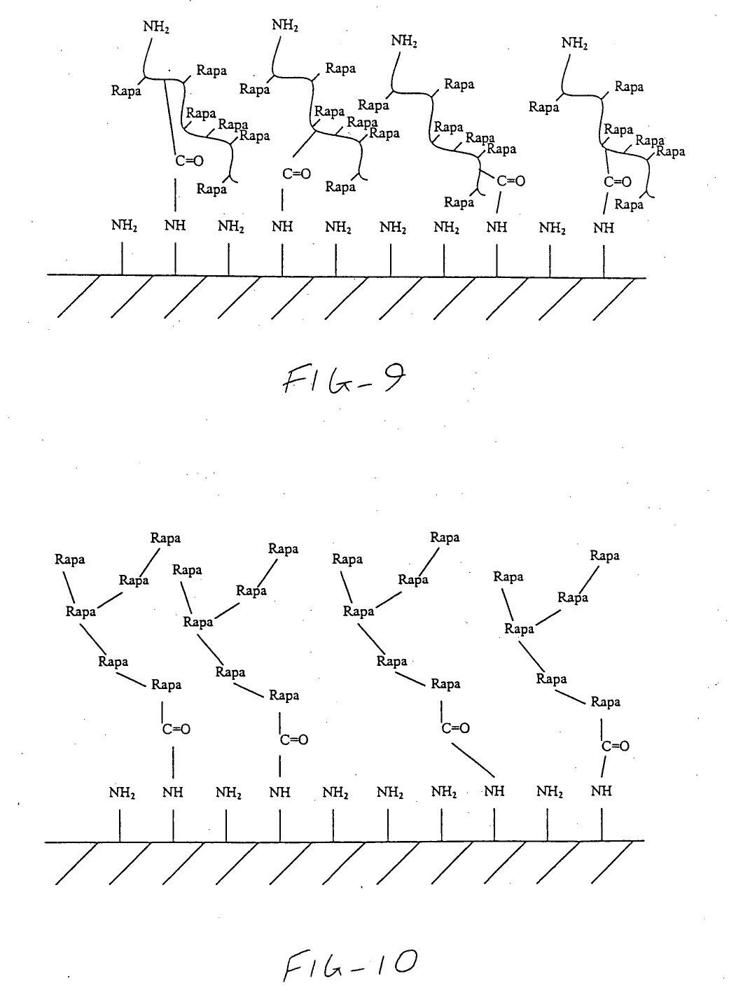

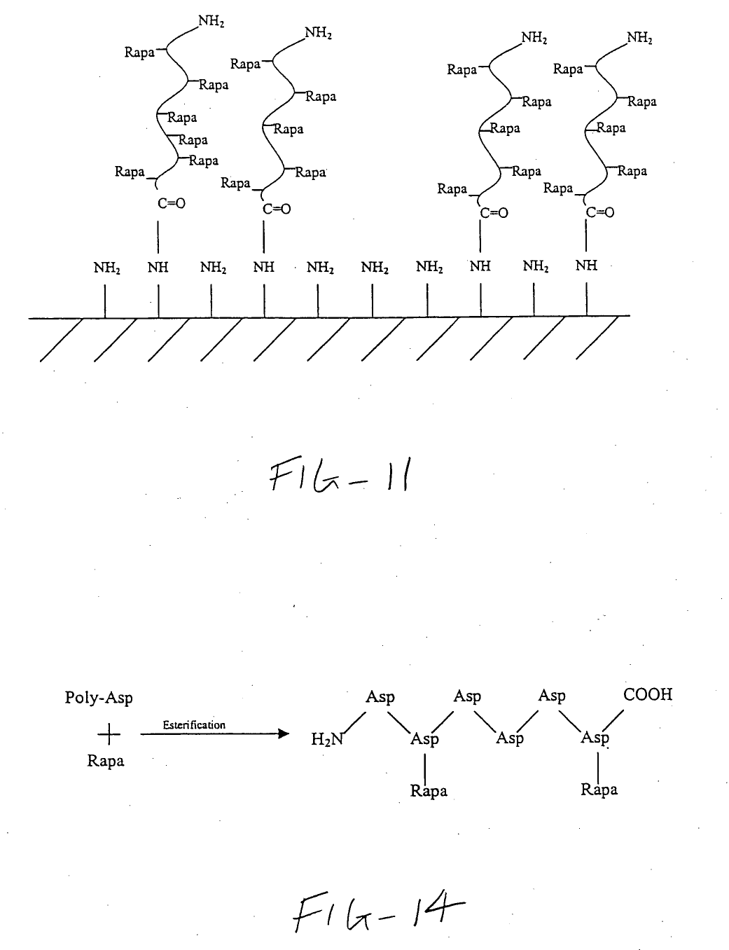

Referring now to FIGS. 7-11, a stent 30 having a surface 32 will typically be composed of a metal, such as 316 stainless steel. Such stents are commercially available and include the BX-Velocity™ stent available from Cordis Corporation, Miami, Fla. The surface 32 of such stainless steel stents may be derivatized to have active binding sites. For example, the surface may undergo plasma deposition of allylamine to generate free amines on the surface 32, as shown in FIG. 8. The amines provide binding sites which would otherwise be absent in the metal-oxide layer normally present on the stent surface. The amines can then be reacted using a variety of chemistries to form amide or other linkages. In particular, the amide linkages can be formed with the carboxy terminals present on any of the rapamycin compositions described above. In particular, in FIG. 9, binding of the rapamycin backbone compositions is illustrated. Polypeptide-rapamycin conjugates are anchored to reactive amine surface via side chain carboxylates on amino acids such as aspartate (polypeptide shown as curved line, rapamycin shown as “rapa,” and metal surface shown as hatched line). In FIG. 10, binding of the polymerized rapamycin compositions is illustrated. Directly polymerized rapamycin derivatives are anchored to reactive amine surface via linker carboxylates (rapamycin shown as “rapa” and metal surface shown as hatched line).

The following examples are offered by way of illustration, not by way of limitation.

### EXPERIMENTAL

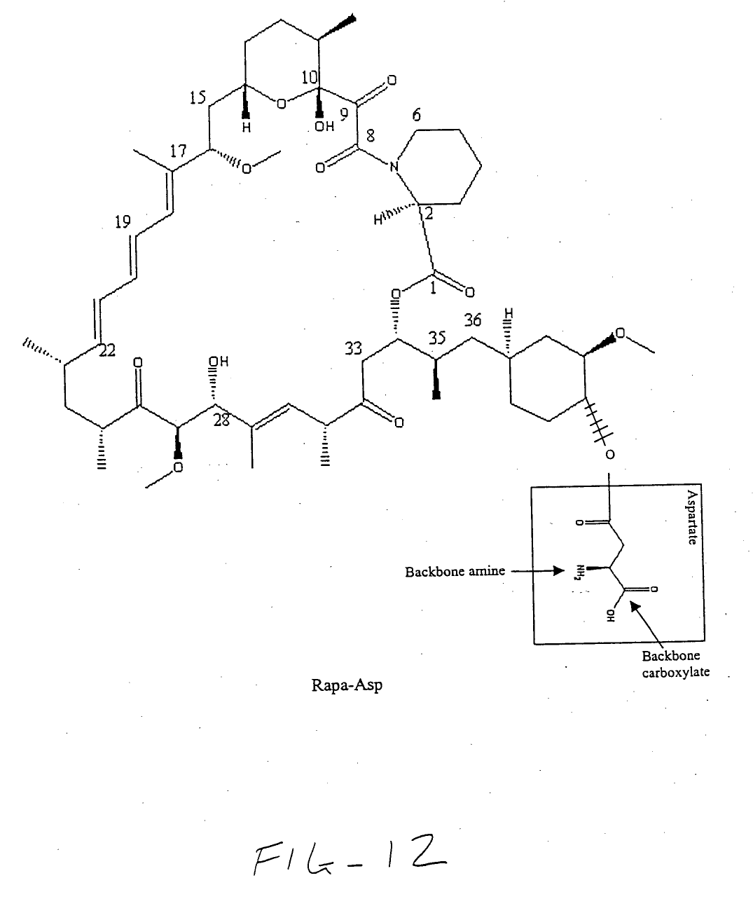

1. Polyaspartate with rapamycin ester side chains. See, FIG. 12. Sodium polyaspartate (Aquadew SPA-30, Ajinomoto, Tokyo, Japan) is reacted with rapamycin in the presence of a sulfuric acid catalyst using standard methods. De Carvalho, M. G. S. et al. Identification of Phosphorylation sites of human 85-kda cytosolic phospholipase A2 expressed in insect cells and present in human monocytes. 1996. J. Biol. Chem 271(12):6987-97. Free carboxylic acid termini on the sodium polyaspartate react with the free hydroxyl at position 42 of rapamycin to form an ester linkage which is degradable in aqueous environments under physiologic conditions. Additionally, the amide linkages of the polyaspartate backbone can be degraded in vivo either by proteases or by non-enzymatic hydrolysis. In this way, multiple rapamycins are added to a single backbone of the polyaspartate. The degree of saturation of rapamycin on the polyaspartate can be controlled by varying the reaction conditions, such as the concentration of rapamycin, the concentration of sodium polyaspartate, the concentration of the sulfuric acid catalyst, the duration of the reaction, the temperature of the reaction, and the like, as is well-known to one skilled in the art.

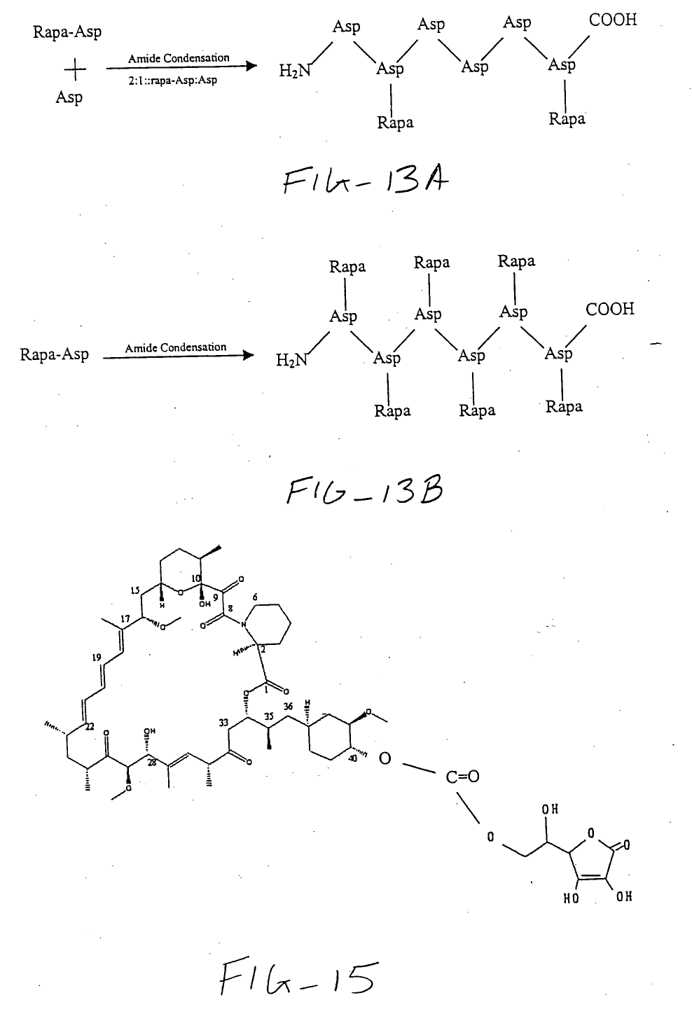

Polyaspartate having rapamycin ester side chains could also be formed by first forming rapamycin esters with aspartate monomers. The rapamycin ester aspartate monomers could then be polymerized by forming amide linkages between the aspartates. The number of rapamycins incorporated in each polyaspartate form can be controlled by reacting the rapamycin derivatized aspartates with native or otherwise derivatized aspartates. Rapamycin (rapa)-aspartate (Asp) conjugates may be polymerized in the presence of (FIG. 13A) or absence (FIG. 13B) of native (or other conjugated aspartate). The ratio of native to conjugated aspartate in the polymer will be the same as that in the reaction volume, so the degree of rapamycin saturation in the resulting polymer can be determined in the protocol of FIG. 13A. Esterification of pre-polymerized aspartate (polyaspartate) with rapamycin is shown in FIG. 14.

2. Polylysine with rapamycin side chains. Polylysine (p-1399, Sigma Chemical Company, St. Louis, Mo.) has free primary amines as termini on each side chain. The free primary amines are converted to free thiols using Traut's reagent (Pierce Endogen, Rockford, Ill.) under standard conditions. The reaction can be controlled to convert any number of the side chain amines from a minimum of three to all. The thiol side chains are then covalently bound to the free hydroxyl at position 42 of rapamycin using PMPI (Pierce Endogen), according to the manufacturer's recommendations. PMPI is a heterobifunctional linker which joins free hydroxyls and free thiols.

The PMPI linker could be used with other poly (amino acids) or polypeptides which have free thiols in their side chains.

3. Polylysine with amide-ester link rapamycin. The free amines of polylysine are reacted with the free hydroxyl at position 42 of rapamycin using carbonic acid or bicarbonate. This reaction is described in U.S. Pat. No. 6,371,975 and generates a mixed polymer of rapamycin and a free amine-rich peptide with mixed ester-amide linkages. The ester-amide linkages are degradable.

4. Rapamycin bound to a polyethylene glycol (PEG) backbone. As described in U.S. Patent Publication No. US 2002/0055518A1, free thiols can be generated on rapamycin. The free thiols on the rapamycin may then be reacted with PEG to produce a composition according to the present invention using a linker such as PMPI which joins free hydroxyls and sulfhydryls. Alternately, carbonic acid or bicarbonate can be used to form a mixed ester between the hydroxyls of rapamycin and the hydroxyls of PEG using methods described in U.S. Pat. No. 6,371,975.

5. Rapamycin on branched polyethylene glycol (PEG) backbone. Free hydroxyls on a branched polyethylene glycol molecule can be reacted with free hydroxyls on rapamycin, typically at position 42, to form esters. Suitable PEG molecules will have three to four branches each and molecular weights below 10,000. Such PEG materials are available from Shearwater Polymers, (Huntsville, Ala., USA), Nippon-Ho (Japan), and Polymer Source (Canada). The resulting mixed diester linkages are degradable in aqueous environments under physiologic conditions.

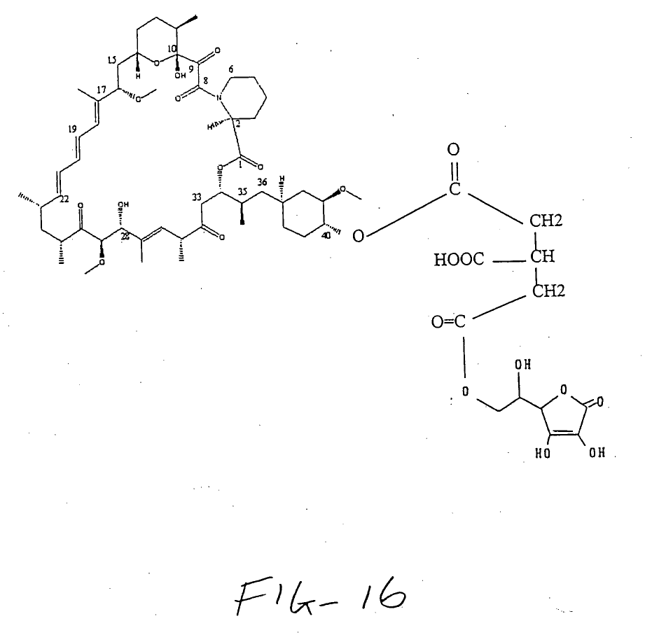

6. Polymerized rapamycin ascorbic acid conjugates. Rapamycin is reacted with ascorbic acid to produce an ester linkage according to well-known techniques. US Patent Publication Nos. US 2002/0031557 A1; US 2002/0037314 A1; and US 2001/0041193 A1; and Maugard, T., et al. (2000). Studies of vitamin ester synthesis by lipase-catalyzed transesterification in organic media. Biotechnol. Prog. 16(3):358-362. The rapamycin ascorbic acid conjugates are then polymerized via free hydroxyls on the ascorbic acid and/or rapamycin or anchored to a polymerizable backbone using the techniques described above. Ascorbic acid, also known as vitamin C, is an anti-oxidant which may provide benefits when the compositions of the present invention are used for hyperplasia inhibition or other purposes. Rapamycin-ascorbic acid hybrid produced from carbonic acid esterification is shown in FIG. 15. Remaining fee hydroxyls can be derivatized or reacted to add polymerizable groups. Simple rapamycin-ascorbic acid hybrid from citric acid esterification is shown in FIG. 16. Free acid groups can react with hydroxyls from adjacent hybrids to cross-link directly or can be reacted with a separate backbone.

Rapamycin may be derivatized with other materials which are useful for polymerization and which also provide other functionalities in the polymerized molecules. For example, rapamycin may be derivatized with vitamin E, various nitric oxide donors, anti-angiogenic agents, such as angiostatin, HMAG CoA reductase inhibitors, and the like. The resulting heterobifunctional rapamycin monomers may then be polymerized to produce the compositions of the present invention using known techniques. Useful derivatized rapamycin molecules are described in U.S. Pat. Nos. 5,985,890; 5,780;462; 5,504,091; 5,411,967; 5,391,730; 5,389,639; 5,385,909; 5,385,908; 5,362,718; 5,302,584; 5,258,389; 5,233,036; 5,221,670; 5,177,203; 5,162,333; 5,151,413; 5,130,307; 5,120,842, 5,118,678; 5,118,677; 5,100,883; 5,023,264; 4,650,803; 4,401,653; and 4,316,885, the full disclosures of which are incorporated herein by reference.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A composition of matter comprising a linked plurality of molecules which specifically bind to the mammalian target of rapamycin (mTOR).

2. A composition of matter as in claim 1, wherein the molecules are selected from the group consisting of rapamycin, rapamycin hybrids, CCI-779, RAD-001, SDZ Rad (Everolimus), FK506 (Tacrolimus), ASM 981 (Pimecrolimus), Wortmannin, and Tumistatin.

3. A composition as in claim 2, having from 3 to 10⁶ molecules linked.

4. A composition as in claim 3, having from 5 to 10⁵ molecules linked.

5. A composition as in claim 4, having from 7 to 5×10⁴ molecules linked.

6. A composition of matter as in any of claim 1, wherein the molecules are linked via attachment to a backbone.

7. A composition of matter as in claim 6, wherein the molecules comprise rapamycin molecules which have been derivatized with linking moieties and wherein the rapamycin molecules are covalently bound through the moieties to the backbone.

8. A composition of matter as claim 7, wherein the linking moieties are bound to the rapamycin molecules at sites which do not sterically interfere with the active sites of rapamycin so that rapamycin retains its activity when attached to the backbone.

9. A composition of matter as in claim 7, wherein the linking moieties are bound to the rapamycin molecules at sites which sterically interfere with the active sites of rapamycin so that rapamycin activity is inhibited while the rapamycin remains attached to the backbone and restored when the rapamycin is released from the backbone.

10. A composition of matter as in claim 6, wherein the backbone degrades under preselected conditions to release the rapamycin molecules.

11. A composition of matter as in claim 7, wherein the linking moieties lyse under preselected conditions to release the rapamycin molecules from the backbone.

12. A composition of matter as in claim 7, wherein the backbone comprises a poly (amino acid).

13. A composition of matter as in claim 12, wherein the backbone is polyaspartate, wherein rapamycin is covalently attached via an ester linkage between a free carboxylic acid on the aspartate side chain to a free hydroxyl at position 42 of rapamycin.

14. A composition of matter as in claim 12, wherein the backbone is polylysine, wherein rapamycin is covalently attached via a heterobifunctional linker between a free thiol on the lysine to a free hydroxyl at position 42 of rapamycin.

15. A composition of matter as in claim 12, wherein the backbone is polylysine, wherein rapamycin is covalently attached via an amide-ester linkage between a free amine on the lysine to a free hydroxyl at position 42 of rapamycin.

16. A composition of matter as in claim 12, wherein the backbone is polylysine, wherein rapamycin is covalently attached via a disulfide linkage through a free thiol introduced to the rapamycin.

17. A composition of matter as in claim 6, wherein the backbone comprises polyethylene glycol (PEG), wherein the molecules comprise rapamycin attached to the PEG by ester linkages between free hydroxyls on the PEG and on the rapamycin.

18. A composition of matter as in any of claim 1, wherein the molecules are polymerized.

19. A composition of matter as in claim 18, wherein the molecules comprise rapamycin molecules which have been derivatized with linking moieties and wherein the rapamycin molecules are polymerized through the linking moieties.

20. A composition of matter as in claim 19, wherein the linking moieties are bound to the rapamycin molecules at sites which do not sterically interfere with the active sites of rapamycin so that rapamycin retains its activity when polymerized.

21. A composition of matter as in claim 19, wherein the linking moieties are bound to the rapamycin molecules at sites which sterically interfere with the active sites of rapamycin so that rapamycin activity is inhibited while the rapamycin remains polymerized and restored when the rapamycin is released.

22. A composition of matter as in claim 19, wherein the linking moieties lyse under preselected conditions.

23. A composition of matter as in claim 19, wherein the linking moieties comprise ascorbic acid attached to the rapamycin molecules via an ester linkage.

24. An implantable prosthesis comprising:
a structure having a surface; and
linked pluralities of molecules which specifically bind to the mammalian target of rapamycin (mTOR) present on the surface.

25. An implantable prosthesis as in claim 24, wherein the structure comprises a vascular prosthesis or stent implantable in a blood vessel.

26. An implantable prosthesis as in claim 24, wherein the linked pluralities are covalently attached to the surface.

27. An implantable prosthesis as in claim 24, wherein the linked plurality of molecules comprise molecules which are selected from the group consisting of rapamycin, rapamycin hybrids, CCI-779, RAD-001, SDZ Rad (Everolimus), FK506 (Tacrolimus), ASM 981 (Pimecrolimus), Wortmannin, and Tumistatin.

28. An implantable prosthesis as in claim 27, having from 3 to 106 molecules linked.

29. An implantable prosthesis as in claim 28, having from 5 to 10⁵ molecules linked.

30. An implantable prosthesis as in claim 29, having from 7 to 5×10⁴ molecules linked.

31. An implantable prosthesis as in any of claim 24, wherein the molecules are linked via attachment to a backbone.

32. An implantable prosthesis as in claim 31, wherein the molecules comprise rapamycin molecules which have been derivatized with linking moieties and wherein the rapamycin molecules are covalently bound through the moieties to the backbone.

33. An implantable prosthesis as claim 32, wherein the linking moieties are bound to the rapamycin molecules at sites which do not sterically interfere with the active sites of rapamycin so that rapamycin retains its activity when attached to the backbone.

34. An implantable prosthesis as in claim 32, wherein the linking moieties are bound to rapamycin molecules at sites which sterically interfere with the active sites of rapamycin so that rapamycin activity is inhibited while the rapamycin remains attached to the backbone and restored when the rapamycin is released from the backbone.

35. An implantable prosthesis as in claim 31, wherein the backbone degrades under preselected conditions to release the rapamycin molecules.

36. An implantable prosthesis as in claim 32, wherein the linking moieties lyse under preselected conditions to replace the rapamycin molecules from the backbone.

37. An implantable prosthesis as in claim 32, wherein the backbone comprises a poly (amino acid).

38. An implantable prosthesis as in claim 37, wherein the backbone is polyaspartate, wherein rapamycin is covalently attached via an ester linkage between a free carboxylic acid on the aspartate side chain to a free hydroxyl at position 42 of rapamycin.

39. An implantable prosthesis as in claim 37, wherein the backbone is polylysine, wherein rapamycin is covalently attached via a heterobifunctional linker between a free thiol on the lysine to a free hydroxyl at position 42 of rapamycin.

40. An implantable prosthesis as in claim 37, wherein the backbone is polylysine, wherein rapamycin is covalently attached via an amide-ester linkage between a free amine on the lysine to a free hydroxyl at position 42 of rapamycin.

41. An implantable prosthesis as in claim 37, wherein the backbone is polylysine, wherein rapamycin is covalently attached via a disulfide linkage through a free thiol introduced to the rapamycin.

42. An implantable prosthesis as in claim 31, wherein the backbone comprises polyethylene glycol (PEG), wherein the molecules comprise rapamycin attached to the PEG by ester linkages between free hydroxyls on the PEG and on the rapamycin.

43. An implantable prosthesis as in any of claim 24, wherein the molecules are polymerized.

44. An implantable prosthesis as in claim 43, wherein the molecules comprise rapamycin molecules which have been derivatized with linking moieties and wherein the rapamycin molecules are polymerized through the linking moieties.

45. An implantable prosthesis as in claim 44, wherein the linking moieties are bound to the rapamycin molecules at sites which do not sterically interfere with the active sites of rapamycin so that rapamycin retains its activity when polymerized.

46. An implantable prosthesis as in claim 44, wherein the linking moieties are bound to the rapamycin molecules at sites which sterically interfere with the active sites of rapamycin so that rapamycin activity is inhibited while the rapamycin remains polymerized and restored when the rapamycin is released.

47. An implantable prosthesis as in claim 44, wherein the linking moieties lyse under preselected conditions.

48. An implantable prosthesis as in claim 44, wherein the linking moieties comprise ascorbic acid attached to the rapamycin molecules via an ester linkage.

49. A method for preparing a linked plurality of molecules which specifically bind to the mammalian target of rapamycin (mTOR), said method comprising:
providing a backbone molecule; and
binding the plurality of molecules to the backbone molecule.

50. A method as in claim 49, wherein the molecules are selected from the group consisting of rapamycin, rapamycin hybrids, CCI-779, RAD-001, SDZ Rad (Everolimus), FK506 (Tacrolimus), ASM 981 (Pimecrolimus), Wortmannin, and Tumistatin.

51. A method as in claim 50, wherein the plurality consists of from 3 to 10⁶ molecules.

52. A method as in claim 51, wherein the plurality consists of from 5 to 10⁵ molecules.

53. A method as in claim 52, wherein the plurality consists of from 7 to 5×10⁴ molecules.

54. A method as in any of claim 49, wherein the molecules comprise rapamycin molecules which have been derivatized with linking moieties and wherein the rapamycin molecules are covalently bound through the moieties to the backbone.

55. A method as in claim 54, wherein the linking moieties are bound to the rapamycin molecules at sites which do not sterically interfere with the active sites of rapamycin so that rapamycin retains its activity when attached to the backbone.

56. A method as in claim 54, wherein the linking moieties are bound to rapamycin molecules at sites which sterically interfere with the active sites of rapamycin so that rapamycin activity is inhibited while the rapamycin remains attached to the backbone and restored when the rapamycin is released from the backbone.

57. A method as in claim 54, wherein the backbone degrades under preselected conditions to release the rapamycin molecules.

58. A method as in claim 54, wherein the linking moieties lyse under preselected conditions to replace the rapamycin molecules from the backbone.

59. A method as in claim 54, wherein the backbone comprises a poly (amino acid).

60. A method as in claim 59, wherein the backbone is polyaspartate, wherein rapamycin is covalently attached via an ester linkage between a free carboxylic acid on the aspartate side chain to a free hydroxyl at position 42 of rapamycin.

61. A method as in claim 59, wherein the backbone is polylysine, wherein rapamycin is covalently attached via a heterobifunctional linker between a free thiol on the lysine to a free hydroxyl at position 42 of rapamycin.

62. A method as in claim 59, wherein the backbone is polylysine, wherein rapamycin is covalently attached via an amide-ester linkage between a free amine on the lysine to a free hydroxyl at position 42 of rapamycin.

63. A method as in claim 59, wherein the backbone is polylysine, wherein rapamycin is covalently attached via a disulfide linkage through a free thiol introduced to the rapamycin.

64. A method as in claim 59, wherein the backbone comprises polyethylene glycol (PEG), wherein the molecules comprise rapamycin attached to the PEG by ester linkages between free hydroxyls on the PEG and on the rapamycin.

65. A method for preparing a linked plurality of molecules which specifically bind to the mammalian target of rapamycin, said method comprising:
polymerizing the molecules.

66. A method as in claim 65, wherein the plurality consists of from 3 to 10⁶ molecules.

67. A method as in claim 66, wherein the plurality consists of from 5 to 10⁵ molecules.

68. A method as in claim 67, wherein the plurality consists of from 7 to 5×10⁴ molecules.

69. A method as in claim 65, wherein the molecules comprise rapamycin.

70. A method as in claim 69, wherein polymerizing comprises:
derivatizing the rapamycin molecules with a polymerizable moiety; and
polymerizing the polymerizable moieties to covalently bind the rapamycin molecules via the moieties.

71. A method as in claim 70, wherein the linking moieties are bound to the rapamycin molecules at sites which do not sterically interfere with the active sites of rapamycin so that rapamycin retains its activity when polymerized.

72. A method as in claim 70, wherein the linking moieties are bound to the rapamycin molecules at sites which sterically interfere with the active sites of rapamycin so that rapamycin activity is inhibited while the rapamycin remains polymerized and restored when the rapamycin is released.

73. A method as in claim 70, wherein the polymerized moieties lyse under preselected conditions.

74. A method as in claim 70, wherein the polymerized moieties comprise ascorbic acid attached to the rapamycin molecules via an ester linkage.

75. A method as in claim 70, wherein the polymerizable moiety comprises ascorbic acid.

76. A method for modifying an implantable prosthesis, said method comprising:
providing an implantable prosthesis having a surface; and
binding linked pluralities of molecules which specifically bind to the mammalian target of rapamycin (mTOR).

77. A method as in claim 77, wherein the implantable prosthesis comprises a vascular prosthesis or stent implantable in a blood vessel.

78. A method as in claim 77, wherein binding comprises covalently attaching linked pluralities of rapamycin to the surface.

79. A method as in claim 78, wherein binding comprises generating free amines on the surface and forming an amide linkage to a carboxy moiety in the linked pluralities of rapamycin.

80. A method as in claim 78, wherein the linked pluralities of rapamycin have from 3 to 10⁶ molecules linked.

81. A method as in claim 79, wherein the linked pluralities of rapamycin have from 5 to 10⁵ molecules linked.

82. A method as in claim 80, wherein the linked pluralities of rapamycin have from 7 to 5×10⁴ molecules linked.

83. A method as in any of claim 78, wherein the linked pluralities of rapamycin are linked via attachment to a backbone.

84. A method as in claim 83, wherein the molecules comprise rapamycin molecules which have been derivatized with linking moieties and wherein the rapamycin molecules are covalently bound through the moieties to the backbone.

85. A method as claim 84, wherein the linking moieties are bound to the rapamycin molecules at sites which do not sterically interfere with the active sites of rapamycin so that rapamycin retains its activity when attached to the backbone.

86. A method as in claim 84, wherein the linking moieties are bound to rapamycin molecules at sites which sterically interfere with the active sites of rapamycin so that rapamycin activity is inhibited while the rapamycin remains attached to the backbone and restored when the rapamycin is released from the backbone.

87. A method as in claim 83, wherein the backbone degrades under preselected conditions to release the rapamycin molecules.

88. A method as in claim 83, wherein the linking moieties lyse under preselected conditions to replace the rapamycin molecules from the backbone.

89. A method as in claim 83, wherein the backbone comprises a poly (amino acid).

90. A method as in claim 89, wherein the backbone is polyaspartate, wherein rapamycin is covalently attached via an ester linkage between a free carboxylic acid on the aspartate side chain to a free hydroxyl at position 42 of rapamycin.

91. A method as in claim 89, wherein the backbone is polylysine, wherein rapamycin is covalently attached via a heterobifunctional linker between a free thiol on the lysine to a free hydroxyl at position 42 of rapamycin.

92. A method as in claim 89, wherein the backbone is polylysine, wherein rapamycin is covalently attached via an amide-ester linkage between a free amine on the lysine to a free hydroxyl at position 42 of rapamycin.

93. A method as in claim 89, wherein the backbone is polylysine, wherein rapamycin is covalently attached via a disulfide linkage through a free thiol introduced to the rapamycin.

94. A method as in claim 83, wherein the backbone comprises polyethylene glycol (PEG), wherein the molecules comprise rapamycin attached to the PEG by ester linkages between free hydroxyls on the PEG and on the rapamycin.

95. A method as in any of claim 78, wherein the molecules are polymerized.

96. A method as in claim 95, wherein the molecules comprise rapamycin molecules which have been derivatized with linking moieties and wherein the rapamycin molecules are polymerized through the linking moieties.

97. A method as in claim 96, wherein the linking moieties are bound to the rapamycin molecules at sites which do not sterically interfere with the active sites of rapamycin so that rapamycin retains its activity when polymerized.

98. A method as in claim 96, wherein the linking moieties are bound to the rapamycin molecules at sites which sterically interfere with the active sites of rapamycin so that rapamycin activity is inhibited while the rapamycin remains polymerized and restored when the rapamycin is released.

99. A method as in claim 96, wherein the linking moieties lyse under preselected conditions.

100. A method as in claim 96, wherein the linking moieties comprise ascorbic acid attached to the rapamycin molecules via an ester linkage.

101. A composition as in any of claim 1, further comprising an unlinked ascorbic acid moiety.
